(19) United States
(12) Patent Application Publication
(10) Pub. No.: US 2023/0355938 A1
(43) Pub. Date: Nov. 9, 2023

(54) DILATOR FOR VASCULAR ACCESS SYSTEMS, AND ASSOCIATED DEVICES AND METHODS

(71) Applicant: Inari Medical, Inc., Irvine, CA (US)

(72) Inventors: Benjamin Edward Merritt, San Clemente, CA (US); Parker Ozenne, Aliso Viejo, CA (US); Jacob F. Louw, Carlsbad, CA (US); Kali Wen-Tseng Slaughler, Santa Ana, CA (US); Kendall Anne Ruggles, New Port Beach, CA (US)

(21) Appl. No.: 18/311,468

(22) Filed: May 3, 2023

Related U.S. Application Data
(60) Provisional application No. 63/338,266, filed on May 4, 2022.
(60) Provisional application No. 63/397,649, filed on Aug. 12, 2022.

(51) Int. Cl. A61M 29/00; A61M 39/22

(52) U.S. Cl. CPC A61M 29/00; A61M 39/22

(57) ABSTRACT

Disclosed herein are dilators for vascular access systems, and associated devices and methods. In some embodiments, a dilator includes an elongate body having a diameter that is generally constant or uniform along the length of the body. The diameter can be sized to correspond with a catheter of a vascular access system, such that the dilator can be positioned within the catheter with little or no gaps or spacing between the dilator and an interior of the catheter. Additionally, or alternatively, the body of the dilator can include multiple body regions, each having one or more respective mechanical properties. In these and other embodiments, the dilator can be configured to interface with at least a portion of the vascular access system. For example, the dilator can include one or more notches configured to couple the dilator to the vascular access system via a valve of the vascular access system.

### CROSS-REFERENCE TO RELATED APPLICATION(S)

This application claims benefit of U.S. Patent Application No. 63/338,266, filed May 4, 2022 and U.S. Patent Application No. 63/397,649, filed Aug. 12, 2022, each of which is herein incorporated by reference in its entirety.

### TECHNICAL FIELD

The present technology generally relates to dilators for vascular access systems, and associated devices and methods.

### BACKGROUND

Thrombosis is the local coagulation or clotting of the blood in a part of the circulatory system, and a thrombus is a blood clot formed in situ within the vascular system. A venous thrombus is a blood clot that forms within a vein. A common type of venous thrombosis is a deep vein thrombosis (DVT), which is the formation of a blood clot within a deep vein (e.g., predominantly in the legs). Nonspecific signs of a thrombosis may include pain, swelling, redness, warmness, and engorged superficial veins.

If the thrombus breaks off (embolizes) and flows towards the lungs, it can become a life-threatening pulmonary embolism (PE) (e.g., a blood clot in the lungs). In addition to the loss of life that can arise from PE, DVT can cause significant health issues such as post thrombotic syndrome, which can cause chronic swelling, pressure, pain, and ulcers due to valve and vessel damage. Further, DVT can result in significant health-care costs either directly or indirectly through the treatment of related complications and inability of patients to work.

Existing methods for treating DVT and PE often involve treating the DVT or PE with a catheter system that is advanced through the patient's vasculature, such as along a venous access path. Such catheter systems—especially those having a large size (e.g., greater than 16 French)—often include a navigation component, such as a smaller catheter or a dilator, that may be used to dilate the patient's vasculature and guide a large catheter to a target treatment site. For example, the larger catheter may be inserted over the navigation component, using the navigation component as a “rail” to guide the larger catheter to the target treatment site.

However, during advancement of a catheter through the vasculature, the navigation component and/or the catheter can experience considerable force or resistance from the patient's vasculature. In some instances, this may result in practitioners unintentionally injuring patients by over-advancing large catheters along navigation components. Furthermore, the force against the navigation component can cause the navigation component to move or backslide relative to the catheter. If the navigation component bends or flexes relative to the catheter, it can hinder a practitioner's ability to advance the navigation component and catheter together through the patient's vasculature. If the navigation component backslides far enough through the catheter, the edge of the catheter can be exposed and may cause damage to a percutaneous access site or the vasculature of the patient.

### BRIEF DESCRIPTION OF THE DRAWINGS

Many aspects of the present technology can be better understood with reference to the following drawings. The components in the drawings are not necessarily to scale. Instead, emphasis is placed on illustrating clearly the principles of the present disclosure.

FIG. 1 is a partially schematic side view of a vascular access system configured in accordance with the present technology.

FIGS. 2A and 2B are a side view and a side cross-sectional view, respectively, of a dilator configured in accordance with embodiments of the present technology.

FIG. 2C is an enlarged perspective view of region 2C of the dilator shown in FIG. 2A in accordance with embodiments of the present technology.

FIGS. 3A and 3B are side cross-sectional views of the dilator of FIGS. 2A-2C positioned within a valve of the vascular access system of FIG. 1 in accordance with embodiments of the present technology.

FIGS. 4A and 4B are perspective views of the dilator of FIGS. 2A-2C positioned within the vascular access system of FIG. 1 in accordance with embodiments of the present technology.

### DETAILED DESCRIPTION

The present technology is generally directed to navigation components (“dilators”) for vascular access systems, and associated devices and methods. In some embodiments, a dilator includes an elongate body having a diameter that is generally constant or uniform along the length of the body. The diameter can be sized to correspond with a catheter of a vascular access system, such that the dilator can be positioned within the catheter with little or no gaps or spacing between the dilator and the interior of the catheter. In these and other embodiments, the body of the dilator can include multiple body regions, each having one or more respective mechanical properties. In at least some embodiments, for example, the dilator includes a first body region having a first flexibility profile and a second body region having a second flexibility profile greater than the first flexibility profile, such that the second body region is more flexible than the first body region. The second body region can be distal from the first body region, such that the less flexible/stiffer first body region can provide a portion of the dilator against which force can be applied to advance the dilator through the patient's vasculature, and the less stiff/more flexible second body region can provide improved navigation response and/or control while advancing the dilator through the patient's vasculature.

Currently existing dilators may be tapered or sloped to provide flexibility. For example, the diameter of a tapered dilator can decrease toward the tapered dilator's distal tip, with the decreased diameter providing increased flexibility toward the distal tip. However, these tapered dilators can be difficult to secure relative to vascular access devices such as catheters. For example, the tapered shape of these dilators can lead to gaps or spacing between the tapered dilator and the catheter when the tapered dilator is positioned within the catheter, which can allow the tapered dilators to bend or flex within the catheter during use. This bending or flexing can reduce control and/or navigational precision during a procedure. In contrast to the current sloped dilators, the dilators of the present technology can have generally constant or uniform diameters that improve these dilators' fit within vascular access devices, and/or may reduce or prevent unwanted flexing or bending of these dilators during a procedure. Additionally, or alternatively, the respective mechanical properties of the body regions of the dilators can be selected to improve these dilators' flexibility.

Additionally or alternatively, the dilator can be configured to interface with at least a portion of the vascular access system. In some embodiments, the dilator includes one or more notches. Each of the notches can extend at least partially or fully around a longitudinal axis of the body of the dilator, and can be configured to couple the dilator to the vascular access system via a valve of the vascular access system. For example, the valve can include one or more internal components or tethers configured to open and/or close a path through the valve. When the dilator is positioned within the valve with one of the notches aligned with the tether, the tether can be inserted at least partially into the notch to thereby create a resistive force or interference fit that inhibits or prevents further movement of the dilator relative to the valve (and/or movement of the valve relative to the dilator). Each of the notches can be spaced along the length of the dilator such that, when coupled via the valve, each notch can be associated with a different position of the dilator relative to the vascular access system and/or a different length of the dilator that extends beyond the vascular access system. Because each notch can inhibit further movement of the dilator and/or the vascular access system relative to one another, the positions of the one or more notches can be configured to inhibit or prevent over-insertion of the dilator, and/or can provide a user with increased control over an amount of the dilator that extends beyond the vascular access system.

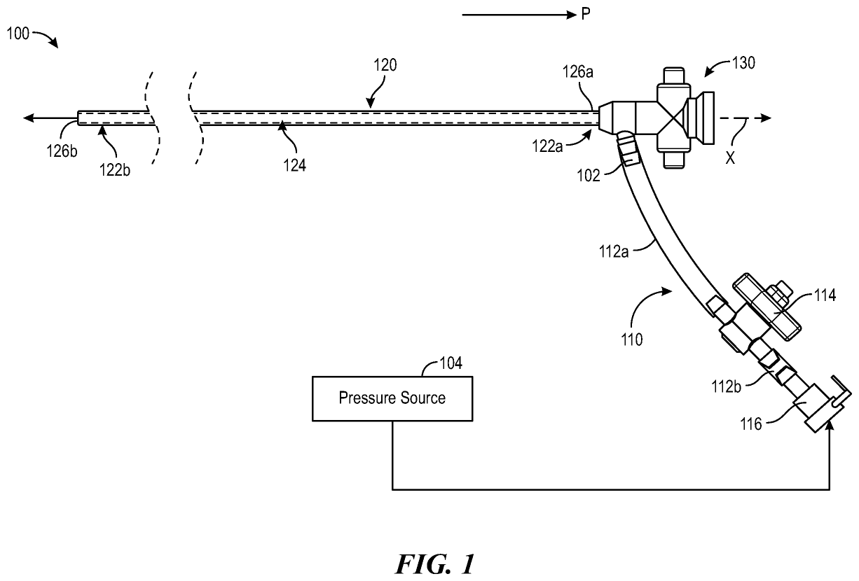

Certain details are set forth in the following description and in FIGS. 1-4B to provide a thorough understanding of various embodiments of the present technology. In other instances, well-known structures, materials, operations, and/or systems often associated with intravascular procedures, clot removal procedures, catheters, and the like are not shown or described in detail in the following disclosure to avoid unnecessarily obscuring the description of the various embodiments of the technology. Those of ordinary skill in the art will recognize, however, that the present technology can be practiced without one or more of the details set forth herein, and/or with other structures, methods, components, and so forth.

The terminology used below is to be interpreted in its broadest reasonable manner, even though it is being used in conjunction with a detailed description of certain examples of embodiments of the technology. Indeed, certain terms may even be emphasized below; however, any terminology intended to be interpreted in any restricted manner will be overtly and specifically defined as such in this Detailed Description section.

The accompanying Figures depict embodiments of the present technology and are not intended to be limiting of its scope unless expressly indicated. The sizes of various depicted elements are not necessarily drawn to scale, and these various elements may be enlarged to improve legibility. Component details may be abstracted in the Figures to exclude details such as position of components and certain precise connections between such components when such details are unnecessary for a complete understanding of how to make and use the present technology. Many of the details, dimensions, angles and other features shown in the Figures are merely illustrative of particular embodiments of the disclosure. Accordingly, other embodiments can have other details, dimensions, angles and features without departing from the present technology. In addition, those of ordinary skill in the art will appreciate that further embodiments of the present technology can be practiced without several of the details described below.

With regard to the terms “distal” and “proximal” within this description, unless otherwise specified, the terms can reference a relative position of the portions of a catheter subsystem with reference to an operator and/or a location in the vasculature. Also, as used herein, the designations “rearward,” “forward,” “upward,” “downward,” and the like are not meant to limit the referenced component to a specific orientation. It will be appreciated that such designations refer to the orientation of the referenced component as illustrated in the Figures; the systems of the present technology can be used in any orientation suitable to the user.

FIG. 1 is a partially schematic side view of a vascular access system 100 (“the system 100”) configured in accordance with embodiments of the present technology. The system 100 can also be referred to as an aspiration assembly, a clot treatment system, a clot removal system, a thrombectomy system, an introducer sheath assembly, and/or the like. In the illustrated embodiment, the system 100 includes a tubing assembly 110 fluidly coupled to a catheter 120 via a valve 130. In some embodiments, the catheter 120 is an elongate member configured to be inserted into and through a patient's vasculature and used to, for example, treat clot material therein. In other embodiments, the catheter 120 can be an introducer sheath configured to be inserted through the skin and tissue tract of the patient to provide an access site through which other components (e.g., other catheters used to treat clot material) can traverse to easily access the vasculature. Accordingly, while referred to as “catheter 120,” the catheter 120 can comprise an introducer sheath, an access sheath, and/or another type of elongate member configured to be inserted through the skin and tissue tract and/or to traverse the vasculature of a patient. In general, the system 100 (i) can include features generally similar or identical to those of the clot treatment systems described in detail in U.S. patent application Ser. No. 16/536,185, filed Aug. 8, 2019, and titled “SYSTEM FOR TREATING EMBOLISM AND ASSOCIATED DEVICES AND METHODS,” which is incorporated herein by reference in its entirety, and/or (ii) can be used to treat/remove clot material from a patient (e.g., a human patient) using any of the methods described in detail therein.

In the illustrated embodiment, the catheter 120 includes a proximal region or portion 122a and a distal tip region or portion 122b opposite the proximal region 122a. The proximal region 122a can include a proximal terminus 126a of the catheter 120, and the distal tip region 122b can include a distal terminus 126b of the catheter 120. The catheter 120 further defines a lumen 124 (shown using dashed-line in FIG. 1) extending entirely therethrough from the proximal region 122a to the distal tip region 122b. The lumen 124 and/or the catheter 120 can at least partially define a longitudinal axis X of the system 100.

The valve 130 is fluidly coupled to the lumen 124 of the catheter 120 and can be integral with or coupled to the proximal region 122a of the catheter 120 such that these components move together. In some embodiments, the valve 130 is a hemostasis valve that is configured to maintain hemostasis during a clot removal procedure by preventing fluid flow in a proximal direction P through the valve 130 as various components such as dilators, delivery sheaths, pull members, guidewires, interventional devices, other aspiration catheters, and so on are inserted through the valve 130 to be delivered through the catheter 120 to a treatment site in a blood vessel. The valve 130 can include a branch or side port 102 configured to fluidly couple the lumen 124 of the catheter 120 to the tubing assembly 110. In some embodiments, the valve 130 can be a valve of the type disclosed in U.S. patent application Ser. No. 16/117,519, filed Aug. 30, 2018, and titled “HEMOSTASIS VALVES AND METHODS OF USE,” which is incorporated herein by reference in its entirety.

In the illustrated embodiment, the tubing assembly 110 fluidly couples the catheter 120 to a pressure source 104, such as a syringe. The pressure source 104 can be configured to generate (e.g., form, create, charge, build-up) a vacuum (e.g., negative relative pressure) and store the vacuum for subsequent application to the catheter 120. The tubing assembly 110 can include one or more tubing sections 112 (individually labeled as a first tubing section 112a and a second tubing section 112b), at least one fluid control device 114 (e.g., a valve), and at least one connector 116 (e.g., a Toomey tip connector) for fluidly coupling the tubing assembly 110 to the pressure source 104 and/or other suitable components. In some embodiments, the fluid control device 114 is a stopcock that is fluidly coupled to (i) the side port 102 of the valve 130 via the first tubing section 112a and (ii) the connector 116 via the second tubing section 112b. The fluid control device 114 is externally operable by a user to regulate the flow of fluid therethrough and, specifically, from the lumen 124 of the catheter 120 to the pressure source 104. In some embodiments, the connector 116 is a quick-release connector (e.g., a quick disconnect fitting) that enables rapid coupling/decoupling of the catheter 120 and the fluid control device 114 to/from the pressure source 104.

During a clot treatment procedure, at least a portion of the system 100, such as the distal terminus 126b and/or distal tip region 122b of the catheter 120, can be inserted through the vasculature of a patient. In some embodiments, the system 100 is inserted through an introducer sheath that traverses the skin and tissue of the patient to provide an access site. When the catheter 120 is positioned at a desired position relative to clot material (e.g., a pulmonary embolism, deep vein thrombosis) within the patient, a user can first close the fluid control device 114 before generating a vacuum in the pressure source 104 by, for example, withdrawing the plunger of a syringe coupled to the connector 116. In this manner, a vacuum is charged within the pressure source 104 (e.g., a negative pressure is maintained) before the pressure source 104 is fluidly connected to the lumen 124 of the catheter 120. To aspirate the lumen 124 of the catheter 120, the user can open the fluid control device 114 to fluidly connect the pressure source 104 to the catheter 120 and thereby apply or release the vacuum stored in the pressure source 104 to the lumen 124 of the catheter 120. Opening the fluid control device 114 instantaneously or nearly instantaneously applies the stored vacuum pressure to the tubing assembly 110 and the catheter 120, thereby generating a suction pulse throughout the catheter 120 that can aspirate the clot material into the catheter 120. In particular, the suction is applied at the distal tip region 122b of the catheter 120 to suck/aspirate at least a portion of the clot material proximate the distal tip region 122b into the lumen 124 of the catheter 120. Additionally, or alternatively, the catheter 120 can act as an introducer sheath and can be inserted through the skin and tissue of a patient and partially into a vessel to provide an access point through which other medical instruments can be delivered and/or otherwise used to treat the patient.

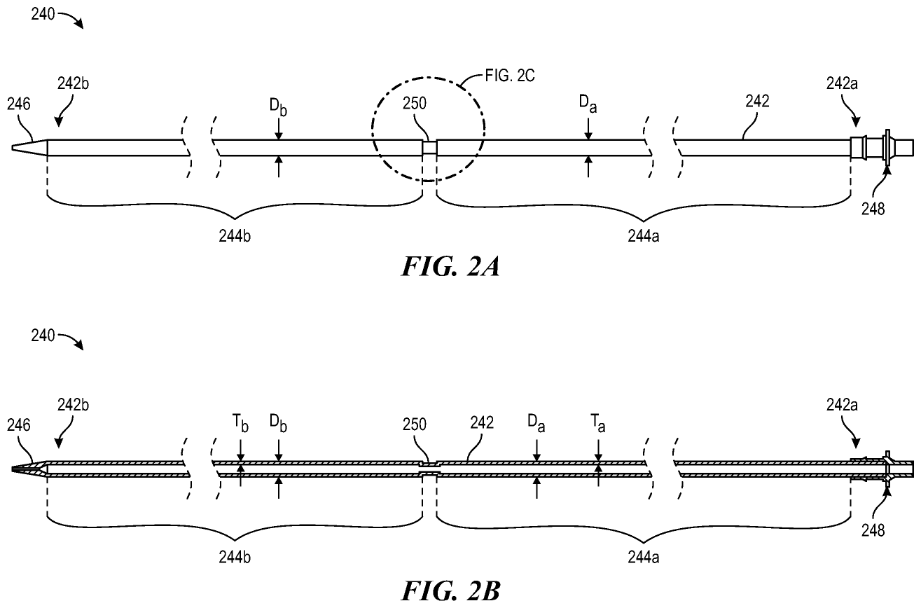

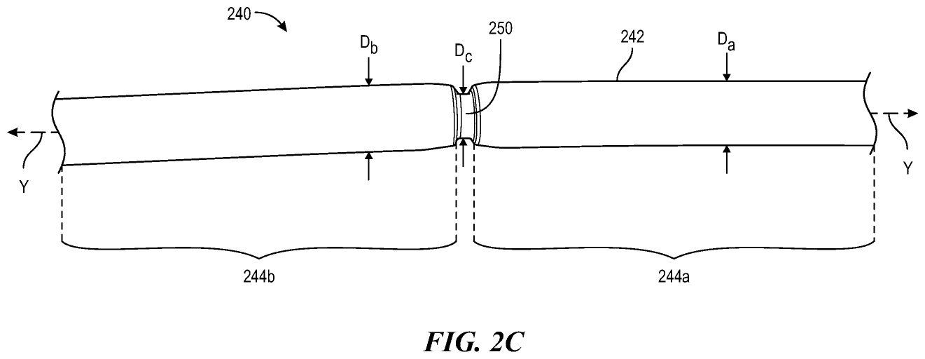

FIGS. 2A and 2B are a side view and a side cross-sectional view, respectively, of a navigation component or dilator 240 configured in accordance with embodiments of the present technology. The dilator 240 is configured to be inserted into and advanced together with the catheter 120 of FIG. 1. The dilator 240 includes a generally elongate body 242 having a proximal region or portion 242a, a distal tip region or portion 242b opposite the proximal region 242a, and one or more body segments or regions 244 (individually identified as a first body region 244a and a second body region 244b) extending at least partially between the proximal region 242a and the distal tip region 242b. The dilator 240 can further include a tip 246 (e.g., an atraumatic tip) in or near the distal tip region 242b, a coupling component 248 in or near the proximal region 242a, and one or more notches 250 (e.g., insertion positioning features). The coupling component 248 can include a twist-lock connector, a luer connector, and/or another suitable coupling component, and can be configured to couple or lock the dilator 240 to a valve of a vascular access system, such as the valve 130 of the system 100 of FIG. 1. In the illustrated embodiment, the tip 246 is distal from the second body region 244b and the coupling component 248 is proximal from the first body region 244a. In other embodiments, the second body region 244b can include all or part of the tip 246, and/or the first body region 244a can include and/or be positioned within all or part of the coupling component 248. The notch 250 is described in further detail below with reference to FIG. 2C.

In some embodiments, the body 242, the tip 246, and/or the coupling component 248 can be hollow, as shown in FIG. 2B, such that a guide wire (not shown) can be positioned within the dilator 240 and used to aid the navigation thereof. In such embodiments, each of the body regions 244 can have a respective thickness T, individually labeled in FIG. 2B as a first thickness Tₐ and a second thickness T_{b}. The first and second thicknesses T_{a-b} can be the same or different, and/or each thickness T_{a-b} may be constant or variable along all or part of the length of the respective body region 244a-b.

The first body region 244a, the second body region 244b, the tip 246, and/or the coupling component 248 can each be formed from one or more polymers, and/or other suitable materials. For example, the first and second body regions 244a-b can be formed from the same or different materials.

Each of the body regions 244a-b can have a respective outer dimension or diameter D, individually labeled as a first diameter Dₐ and a second diameter D_{b}. In some embodiments, the diameters D_{a-b} of the body regions 244a-b can be different from each other and/or vary along their respective lengths, such that the first and second body regions 244a-b, and/or one or more portions thereof, can have different diameters. In the illustrated embodiment, the first and second diameters D_{a-b} are generally similar or identical such that the body 242 of the dilator 240 has a substantially uniform diameter. In these and other embodiments, one or more of the diameters D_{a-b} can correspond to and/or be less than an inner dimension of a catheter, such as an inner diameter of the lumen 124 of the catheter 120 (FIG. 1). Accordingly, the dilator 240 can be positioned within the catheter 120 (e.g., via the valve 130) with at least a portion of the tip 246 extending distally beyond the distal terminus 126b of the catheter 120 (FIG. 1).

Each of the body regions 244 can have one or more respective mechanical properties, such as a hardness, stiffness, durometer, flexibility, rigidity, Young's modulus, density, and/or the like. The mechanical properties (e.g., varying durometer) can give the body regions 244 different flexibility profiles. In some embodiments, individual ones of the body regions 244 can include one or more coils, braids, and/or other structures configured to increase or decrease the flexibility profile, e.g., relative to one or more other body regions 244. In at least some embodiments, the first body region 244a has a first flexibility profile and the second body region 244b has a second flexibility profile greater than the first flexibility profile such that the second body region 244b is less stiff/more flexible than the first body region 244a. For example, in the illustrated embodiment the first body region 244a has a first durometer and the second body region 244b has a second durometer less (e.g., less stiff/more flexible) than the first durometer. In some aspects of the present technology, the less-stiff second body region 244b can improve the navigability of the dilator 240 while traveling through vasculature, while the more-stiff first body region 244a can improve control of and/or force transfer to (e.g., the “pushability” and/or column strength of) the dilator 240 while navigating through the vasculature. Although the dilator 240 shown in FIGS. 2A and 2B includes two body regions 244a-b, in other embodiments the dilator 240 can include more body regions 244, and each of the body regions 244 can have one or more respective mechanical properties greater than, equal to, or less than one or more of the other body regions 244. Accordingly, in some embodiments the dilator 240 includes two or more body regions 244 having the same diameter but different stiffnesses, durometers, flexibilities, etc.

FIG. 2C is an enlarged perspective view of region 2C of FIG. 2A in accordance with embodiments of the present technology. In the illustrated embodiment, the notch 250 defines/comprises a recessed area extending at least partially or fully around the body 242 of the dilator 240, such as circumferentially around a longitudinal axis Y of the body 242. Accordingly, the notch 250 can define a third diameter D_{c} of the dilator 240 that can be less than the first diameter Dₐ and/or the second diameter D_{b}. In the illustrated embodiment, the notch 250 is positioned between the first body region 244a and the second body region 244b. In other embodiments, the notch 250 can be positioned at least partially within one or both of the body regions 244a-b. In these and other embodiments, the dilator 240 can include a plurality of notches 250, individual ones of which can be positioned at least partially between the first and second body regions 244a-b, at least partially within the first body region 244a, at least partially within the second body region 244b, or at another suitable position along the dilator 240.

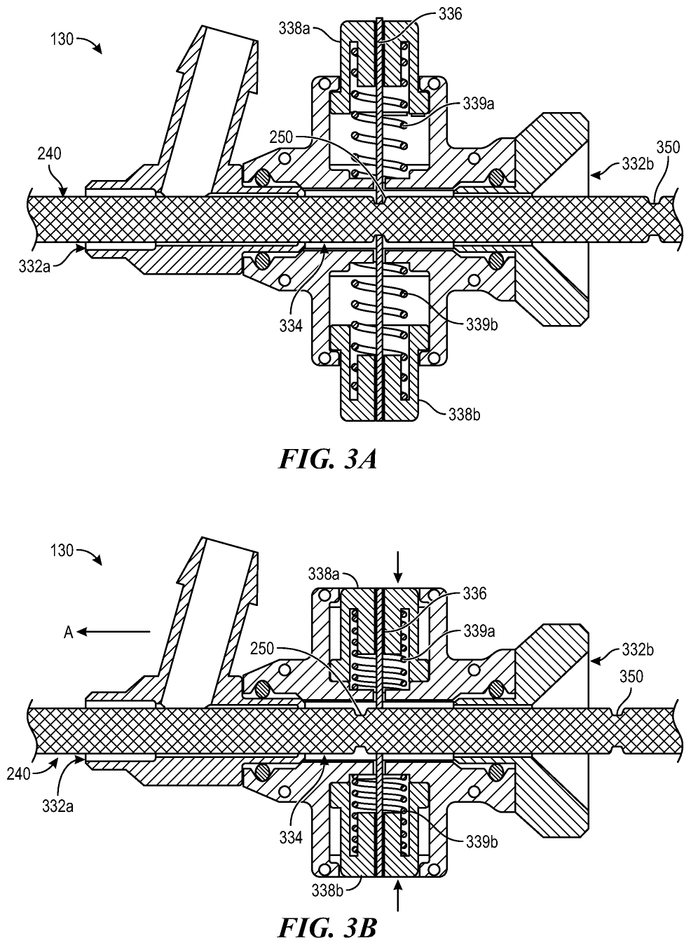

FIGS. 3A and 3B are side cross-sectional views of the dilator 240 positioned within the valve 130 in accordance with embodiments of the present technology. More specifically, in FIG. 3A the dilator 240 is at a first position within the valve 130, and in FIG. 3B the dilator 240 has been moved (e.g., distally) to a second position within the valve 130, as shown by arrow A. In FIGS. 3A and 3B, the dilator 240 includes the notch 250 (“first notch 250”) and an additional second notch 350 positioned proximally from the first notch 250. The second notch 350 can be at least generally similar or identical in structure and/or function to the first notch 250.

Referring to FIGS. 3A and 3B together, the valve 130 includes a first or distal aperture 332a, a second or proximal aperture 332b, and a central lumen 334 extending therebetween. The distal aperture 332a can be fluidly coupled to a catheter, such as the catheter 120 of FIG. 1. The valve 130 further includes a filament or tether 336 operably coupled to one or more actuators or buttons 338 (individually identified as a first button 338a and a second button 338b in FIGS. 3A and 3B). The tether 336 can be configured to open and/or close the central lumen 334 of the valve 130, e.g., to form a substantially fluid-impermeable seal that inhibits or prevents fluid communication between the distal and proximal apertures 332a-b. In the illustrated embodiment, for example, the buttons 338 are biased outwardly by springs 339 (individually identified as a first spring 339a and a second spring 339b in FIGS. 3A and 3B) to a first position shown in FIG. 3A. When the buttons 338a-b are in the first position, the buttons 338a-b draw the tether 336 taught to constrict and seals the central lumen 334. A user can depress the buttons 338 to a second position shown in FIG. 3B against the biasing force of the springs 339 to relax the tether 336 and thereby unconstrict and unseal the central lumen 334. In general, the valve 130 (i) can include at least some features that are generally similar or identical to those of the hemostasis valves described in detail in U.S. Pat. No. 11,000,682, filed Aug. 30, 2018, and titled “HEMOSTASIS VALVES AND METHODS OF USE,” which is incorporated herein by reference in its entirety, and/or (ii) can be used to treat/remove clot material from a patient (e.g., a human patient) using any of the methods described in detail therein.

In some embodiments, the tether 336 is configured to releasably couple the dilator 240 to the valve 130 by interfacing with one of the notches 250, 350. For example, in the illustrated embodiment the tether 336 constricts around the central lumen 334 when the buttons 338a-b are in the first position, as shown in FIG. 3A. Accordingly, when the dilator 240 is inserted through the proximal aperture 332b, the first notch 250 can be positioned in alignment with the tether 336 to thereby allow at least part of the tether 336 to be driven toward and/or into the first notch 250 and create a resistive force/interference fit that couples the dilator 240 to the valve 130. The resistive force/interference fit inhibits or prevents further movement (e.g., proximal and/or distal movement) of the dilator 240 relative to the valve 130.

The dilator 240 can be uncoupled from the valve 130 by actuating/pressing one or more of the buttons 338, as shown in FIG. 3B, to move the tether 336 radially outward away from the longitudinal axis of the central lumen 334 and radially outwardly from/out of the first notch 250. Pressing/actuating one or more of the buttons 338 can thereby allow further movement (e.g., proximal and/or distal movement) of the dilator 240 relative to the valve 130. In some embodiments, the resistive force/interference fit can be overcome by applying increased force to the dilator 240 and/or the valve 130, e.g., to displace the tether 336 from the first notch 250 and allow further movement of the dilator 240 and/or the valve 130 relative to each other without pressing/actuating one or more of the buttons 338. In FIG. 3B, the dilator 240 has been moved further distally through the valve 130 from a proximal position (FIG. 3A) to a more distal position. In some embodiments, the dilator 240 can be advanced further distally until the second notch 350 is positioned in alignment with the tether 336. When the second notch 350 is aligned with the tether 336, the tether 336 can be driven radially into the second notch 350, as described previously with reference to the first notch 250, and thereby again couple the dilator 240 to the valve 130.

Referring to FIGS. 1-3B together, the dilator 240 can be inserted distally through the valve 130 (e.g., via the proximal aperture 332b) and extend fully through the lumen 124 past the distal terminus 126b of the catheter 120, such that the tip 246 of the dilator 240 is positioned beyond the distal terminus 126b of the catheter 120. The notch 250 can be configured to control the position of the dilator 240 relative to the catheter 120, e.g., during insertion. For example, when the dilator 240 is inserted (e.g., distally) through the valve 130 into the catheter 120, the notch 250 can interface with (e.g., releasably couple to) the tether 336 to thereby create a resistive force or interference fit that at least inhibits or prevents further movement (e.g., proximal and/or distal movement) of the dilator 240 relative to the catheter 120. A magnitude of the resistive force/interference fit between the notch 250 and the valve 130 (e.g., the tether 336) can correspond to the difference in size between the third diameter D_{c} and another diameter of a body region of the dilator 240, such as the first diameter Dₐ of the first body region 244a and/or the second diameter D_{b} of the second body region 244b.

Generally, a difference in size between the third diameter D_{c} and a diameter of a body region positioned proximal and adjacent to the notch 250 (e.g., the first diameter Dₐ of the first body region 244a) can be associated with a resistive force/interference fit that inhibits or prevents further distal movement of the dilator 240 through the system 100 (e.g., through the catheter 120) and/or further proximal movement of the system 100 over the dilator 240. Additionally, a difference in size between the third diameter D_{c} and a diameter of a body region positioned distal and adjacent to the notch 250 (e.g., the second diameter D_{b} of the second body region 244b) can be associated with a resistive force/interference fit that at inhibits or prevents further proximal movement of the dilator 240 through the system 100 and/or further distal movement of the system 100 over the dilator 240. For example, if the difference between third diameter D_{c} and the first diameter Dₐ is between about 0.1 mm and about 0.5 mm, the resistive force/interference fit can be relatively slight and partially prevent further (distal) movement of the dilator 240 and/or further (proximal) movement of the system 100. In such embodiments, the resistive force/interference fit may provide a tactile indication or sensation (e.g., a “clicking” sound or sensation perceptible to a user) associated with the relative positions of the dilator 240 and the system 100, and any resistance/interference to further movement may be overcome by applying increased force to the dilator 240 and/or the system 100 to decouple the notch 250 and the valve 130 (e.g., the tether 336). As another example, if the difference between the third diameter D_{c} and the first diameter Dₐ is greater than 0.5 mm, the resistive force/interference fit can be relatively substantial and fully prevent further (distal) movement of the dilator 240 and/or further (proximal) movement of the system 100. In such embodiments, the resistive force/interference fit may be overcome by actuating one or more of the buttons 338 of the valve 130 to release the dilator 240. Dilators with greater diameters can include notches having a greater depth (e.g., corresponding to a greater difference between the third diameter D_{c} and the first and/or second diameters Dₐ, D_{b}) than dilators with a comparatively lesser depth, and greater notch depths are expected to provide increased resistive forces/interference fits than lesser notch depths.

In some embodiments, the magnitude of the resistive force/interference fit is based at least partially on a spring force applied by the valve 130 (e.g., via the springs 339a-b) to the dilator 240. For example, springs with a relatively high spring constant are expected to provide a greater resistive force than springs with a comparatively lower spring constant.

With the dilator 240 positioned within the catheter 120, the dilator 240 and the catheter 120 can together be inserted into a patient (e.g., a human patient) during a clot treatment procedure. For example, the dilator 240 and the catheter 120 can be inserted into and advanced together through a blood vessel of the patient to a target location in the blood vessel. In some embodiments, after the dilator 240 has been inserted through the catheter 120, because the dilator 240 and the catheter 120 can move relative to one another, the catheter 120 can be advanced (e.g., distally, telescopically, etc.) over the dilator 240, e.g., using the dilator 240 as a guide or “rail” to position the catheter 120 at or near the target location. With the catheter 120 positioned at or near the target location, the dilator 240 can then be retracted (e.g., proximally) through the catheter 120 to allow for other intravascular medical devices to be introduced into the patient via the catheter 120 and/or for aspiration of the catheter 120.

In some aspects of the present technology, the uniform diameter and varying stiffness of the dilator 240 is expected to improve the control and advancement characteristics of the dilator 240 and the catheter 120. For example, the uniform diameter of the dilator 240 is expected to better match the inner dimension of the lumen 124 of the catheter 120 which, in turn, can reduce or eliminate gaps or spacing between the dilator 240 and the catheter 120. Reducing/eliminating these gaps can at least partially inhibit or prevent the dilator 240 from flexing or buckling within the catheter 120 when navigating through a patient's vasculature, which can improve control of the dilator's movement during a procedure. Additionally, or alternatively, the uniform diameter of the dilator 240 is expected to improve the durability (e.g., crush resistance) of the catheter 120 when the dilator 240 is positioned within the catheter 120. For example, reducing/eliminating the gaps can also reduce the size of, or eliminate entirely, one or more crush zones associated with the gaps or spacing between the dilator 240 and the catheter 120 and in which the catheter 120 can be crushed or buckled during use. Furthermore, the variable mechanical properties of the dilator's body regions 244a-b can improve the dilator's flexibility and/or response to forces exerted on the dilator 240 (e.g., by a practitioner and/or from the catheter 120) during a procedure. In these and other embodiments, the notch 250 is expected to further improve the control and advancement characteristics of the dilator 240 and/or the system 100. For example, the magnitude of the resistive force/interference fit between the notch 250 and the valve 130 (e.g., the tether 336) can be configured to inhibit or prevent over-insertion of the dilator 240, which may damage a patient's vasculature. Additionally, or alternatively, the tactile feedback or sensation provided by the interference fit between the notch 250 and the valve 130 can provide (e.g., to a user) an indication of the relative position of the dilator 240 within the system 100.

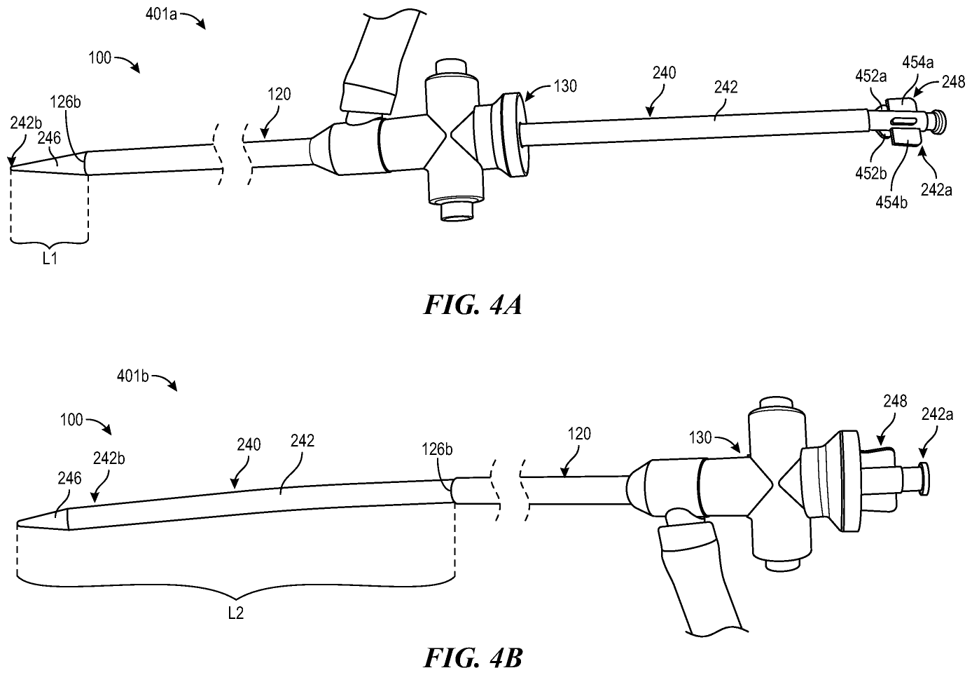

FIGS. 4A and 4B are perspective views of the dilator 240 positioned within the system 100, in accordance with embodiments of the present technology. More specifically, FIG. 4A shows the dilator 240 in a first position or state 401a, and FIG. 4B shows the dilator 240 in a second position or state 401b. In FIGS. 4A and 4B, the coupling component 248 of the dilator 240 includes one or more luer-coupling features 452 (individually identified as a first luer-coupling feature 452a and a second luer-coupling feature 452b in FIGS. 4A and 4B) and/or one or more stopping features 454 (individually identified as a first stopping feature 454a and a second stopping feature 454b). Individual ones of the stopping features 454 can be spaced apart and/or positioned proximally from one or more of the luer-coupling features 452.

Referring to FIG. 4A, the dilator 240 can be inserted through the system 100 until the dilator 240 is coupled to the valve 130 via the notch 250 (FIGS. 2A-3B), as described previously herein with reference to FIGS. 3A and 3B. With the dilator 240 and the system 100 in the first state 401a, a first length L1 of the dilator 240 can extend distally beyond the distal terminus 126b of the catheter 120. In the illustrated embodiment, the first length L1 includes the tip 246 and a portion of the body 242. Additionally, or alternatively, the first length L1 can extend from about 1 cm to about 5 cm beyond the distal terminus 126b. In the first state 401a, the dilator 240 and the catheter 120 can be in a “standard” or “vascular access” mode or configuration in which the catheter 120 and the dilator 240 can be inserted into and/or used to navigate through a patient's vasculature, e.g., to a target location, as described previously herein. The dilator 240 may then be removed from the catheter 120 so that the system 100 can be used to treat and/or remove clot material from within the patient.

Referring to FIG. 4B, the dilator 240 can be advanced from the first state 401a to the second state 401b by advancing the dilator 240 distally through the system 100 and/or retracting the system 100 proximally over the dilator 240. In the second state 401b, a second length L2 of the dilator 240 can extend distally beyond the distal terminus 126b. The second length L2 can be greater than the first length L1. In at least some embodiments, for example, the second length L2 is between about 5 cm and about 20 cm, such as at least 15 cm, or another suitable length therebetween. In the second state 401b, the dilator 240 and the catheter 120 can be in a “rail” or “telescoping delivery” mode or configuration in which the catheter 120 can be advanced into and/or through the patient's vasculature while sliding along and/or otherwise moving over (e.g., telescopically over) the dilator 240. In at least some embodiments, telescopically inserting the catheter 120 over the dilator 240 can include moving from the second state 401b to and/or toward the first state 401a (FIG. 4A). In these and other embodiments, once the dilator 240 and/or the catheter 120 are position at or near a target location, the dilator 240 may then be removed from the catheter 120 the so that the system 100 can be used to treat and/or remove clot material from within the patient.

In some embodiments, one or more of the luer-coupling features 452 (FIG. 4A) can be coupled to the valve 130 in the second state 401b, for example, to at least partially or fully prevent further movement (e.g., unintended movement) of the catheter 120 and/or the dilator 240 relative to each other. Additionally, or alternatively, one or more of the stopping features 454 can contact or abut the valve 130 in the second state 401b, to thereby inhibit or prevent (i) further distal movement of the dilator 240 relative to the system 100 and/or (ii) further proximal movement of the system 100 relative to the dilator 240.

As described in detail above, the dilator 240 can include more than one notch (e.g., including the second notch 350 shown in FIGS. 3A and 3B). Each of the notches can be spaced along the length of the dilator 240 such that, when coupled via the valve 130, each notch can be associated with a different position of the dilator 240 relative to the system 100 and/or a different length of the dilator 240 (e.g., between the first and second lengths L1, L2) that extends beyond the distal terminus 126b. For example, referring to FIGS. 3A-4B together, the second notch 350 is positioned proximal of the first notch 250 such that the dilator extends farther past the distal terminus 126b of the catheter 120 when the valve 130 is coupled to (e.g., locked to) the dilator 240 at the second notch 350.

Because each notch can inhibit further movement of the dilator 240 and/or the system 100 relative to each other, the positions of one or more of the notches can be configured to inhibit or prevent over-insertion of the dilator 240, and/or can provide a user with increased control over the amount of the dilator 240 that extends beyond the distal terminus 126b. Additionally, when the dilator 240 includes multiple notches, the dilator 240 can be configured to be transitioned between more than the two states 401a-b shown in the illustrated embodiment. For example, the dilator 240 can be configured to be transitioned between at least three, four, five, or more different states.

### EXAMPLES

Several aspects of the present technology are set forth in the following examples:
1. A dilator, comprising:
   a tip;
   a coupling component;
   a first body region between the tip and the coupling component, wherein the first body region has a first flexibility profile; and
   a second body region between the first body region and the tip, wherein the second body region has a second flexibility profile greater than the first flexibility profile,
   wherein the first body region and the second body region have a same diameter.
2. The dilator of example 1 wherein the first body region has a first stiffness, wherein the second body region has a second stiffness, and wherein the first stiffness is greater than the second stiffness.
3. The dilator of example 1 or example 2 wherein the first body region has a first durometer, wherein the second body region has a second durometer, and wherein the second durometer is less than the first durometer.
4. The dilator of any of examples 1-3 wherein the first body region comprises a first material, and wherein the second body region comprises a second material different than the first material.
5. The dilator of any of examples 1-4, further comprising a third body region between the tip and the coupling component, wherein the third body region has a third flexibility profile different than the first flexibility profile or the second flexibility profile.
6. The dilator of any of examples 1-5, further comprising a notch positioned between the tip and the coupling component.
7. The dilator of example 6 wherein the notch is positioned between the first body region and the second body region.
8. The dilator of example 6 or example 7 wherein the notch is a first notch, the dilator further comprising a second notch positioned on a proximal or a distal side of the first notch.
9. A vascular access system, comprising:
   a valve having a proximal end and a distal end;
   a catheter defining a lumen and coupled to the valve; and
   a dilator configured to be positioned within the lumen through the valve, wherein the dilator includes—
      a first body region having a first flexibility profile; and
      a second body region having a second flexibility profile less than the first flexibility profile,
      wherein the first body region and the second body region have a same diameter.
10. The vascular access system of example 8 wherein the first body region has a first stiffness, wherein the second body region has a second stiffness, and wherein the first stiffness is greater than the second stiffness.
11. The dilator of example 9 or example 10 wherein the first body region has a first durometer, wherein the second body region has a second durometer, and wherein the second durometer is less than the first durometer.
12. The vascular access system of any of examples 9-11 wherein the first body region comprises a first material, and wherein the second body region comprises a second material different than the first material.
13. The vascular access system of any of examples 9-12, wherein the dilator further includes a notch configured to extend at least partially around a longitudinal axis of the dilator, wherein the valve is configured to be releasably coupled to the dilator via the notch.
14. The vascular access system of example 13 wherein the notch defines a recessed region of the dilator having a notch diameter less than the diameter of the first body region and the second body region.
15. The vascular access system of example 13 or example 14 wherein the valve includes a tether, and wherein the tether is configured to be positioned at least partially within the notch to releasably couple the dilator to the valve.
16. The vascular access system of example 15 wherein the tether is biased in a first direction to releasably couple the dilator to the valve, the valve further comprising a button operably coupled to the tether and configured to move the tether in a second direction, opposite the first direction, to cause the tether to uncouple the dilator from the valve.
17. The vascular access system of any of examples 13-16 wherein the notch is a first notch, and wherein the dilator further includes a second notch positioned on a proximal side or a distal side of the notch.
18. The vascular access system of example 17 wherein the catheter includes a distal terminus, wherein a first length of the dilator extends beyond the distal terminus when the first notch is coupled to the valve, and wherein a second length of the dilator extends beyond the distal terminus when the second notch is coupled to the valve.
19. The vascular access system of example 18 wherein the second length is greater than the first length.
20. A method of treating clot material within a patient, the method comprising:
   positioning a dilator within a vascular access system such that a valve of the vascular access system releasably engages a notch of the dilator to inhibit further movement of the dilator relative to the vascular access system;
   actuating a button of the valve to release the notch from the valve to allow further movement of the dilator relative to the vascular access system; and
   moving the dilator relative to the vascular access system and/or the vascular access system relative to the dilator.
21. The method of example 20 wherein moving the dilator relative to the vascular access system includes advancing the dilator through the vascular access system to increase a length of the dilator that extends beyond a distal terminus of the vascular access system.
22. The method of example 20 wherein moving the vascular access system relative to the dilator includes advancing the vascular access system over the dilator to decrease a length of the dilator that extends beyond a distal terminus of the vascular access system.
23. The method of any of examples 20-22 wherein the notch is a first notch, the method further comprising:
   positioning the dilator within the vascular access system such that the valve releasably engages a second notch of the dilator; and
   applying a force to the dilator and/or the vascular access system to release the second notch from the valve.
24. The method of example 23 wherein applying the force to release the second notch from the valve includes applying the force to release the second notch from the valve without actuating the button.
25. A vascular access system, comprising:
   a dilator extending along a longitudinal axis, wherein the dilator has a varying flexibility profile at least partially along the longitudinal axis, and wherein the dilator defines a notch extending at least partially circumferentially about the longitudinal axis;
   a valve having a proximal end and a distal end; and
   a catheter coupled to the valve and defining a lumen;
   wherein the valve is configured to be releasably coupled to the dilator via the notch to inhibit movement of the dilator relative to the catheter when at least a portion of the dilator is inserted through the valve into the lumen of the catheter.
26. The vascular access system of example 25 wherein the dilator includes a body region having a diameter, and wherein the notch includes a recessed region of the dilator having a notch diameter less than the diameter of the body region.
27. The vascular access system of example 25 or example 26 wherein the valve includes a tether, and wherein the tether is configured to be positioned at least partially within the notch to releasably couple the dilator to the valve.
28. The vascular access system of example 27 wherein the tether is biased in a first direction to releasably couple the dilator to the valve, the valve further comprising a button operably coupled to the tether and configured to move the tether in a second direction, opposite the first direction, to move the tether at least partially out of the notch to uncouple the dilator from the valve.
29. The vascular access system of any of examples 25-28 wherein the notch is a first notch, wherein the portion of the dilator is a first portion, wherein the dilator further defines a second notch extending at least partially circumferentially about the longitudinal axis, and wherein the valve is configured to be releasably coupled to the dilator via the second notch to inhibit movement of the dilator relative to the catheter when at least a second portion of the dilator is inserted through the valve into the lumen of the catheter.
30. The vascular access system of example 29 wherein the catheter includes a distal terminus, wherein a first length of the dilator extends beyond the distal terminus when the first notch is coupled to the valve, and wherein a second length of the dilator extends beyond the distal terminus when the second notch is coupled to the valve.
31. The vascular access system of example 30 wherein the second length is greater than the first length.
32. The vascular access system of any of examples 25-31 wherein the dilator includes a proximal body region having a first flexibility profile and a distal body region having a second flexibility profile greater than the first flexibility profile, wherein the notch is positioned at least partially between the proximal body region and the distal body region.
33. The vascular access system of claim 25 wherein the dilator has a varying durometer at least partially along the longitudinal axis.

The above detailed descriptions of embodiments of the technology are not intended to be exhaustive or to limit the technology to the precise form disclosed above. Although specific embodiments of, and examples for, the technology are described above for illustrative purposes, various equivalent modifications are possible within the scope of the technology as those skilled in the relevant art will recognize. For example, although steps are presented in a given order, alternative embodiments may perform steps in a different order. The various embodiments described herein may also be combined to provide further embodiments.

From the foregoing, it will be appreciated that specific embodiments of the technology have been described herein for purposes of illustration, but well-known structures and functions have not been shown or described in detail to avoid unnecessarily obscuring the description of the embodiments of the technology. Where the context permits, singular or plural terms may also include the plural or singular term, respectively.

Moreover, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. Additionally, the term “comprising” is used throughout to mean including at least the recited feature(s) such that any greater number of the same feature and/or additional types of other features are not precluded. It will also be appreciated that specific embodiments have been described herein for purposes of illustration, but that various modifications may be made without deviating from the technology. Further, while advantages associated with some embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

I/we claim:

1. A dilator, comprising:
a tip;
a coupling component;
a first body region between the tip and the coupling component, wherein the first body region has a first flexibility profile; and
a second body region between the first body region and the tip, wherein the second body region has a second flexibility profile greater than the first flexibility profile,
wherein the first body region and the second body region have a same diameter.

2. The dilator of claim 1 wherein the first body region has a first stiffness, wherein the second body region has a second stiffness, and wherein the first stiffness is greater than the second stiffness.

3. The dilator of claim 1 wherein the first body region has a first durometer, wherein the second body region has a second durometer, and wherein the second durometer is less than the first durometer.

4. The dilator of claim 1 wherein the first body region comprises a first material, and wherein the second body region comprises a second material different than the first material.

5. The dilator of claim 1, further comprising a third body region between the tip and the coupling component, wherein the third body region has a third flexibility profile different than the first flexibility profile or the second flexibility profile.

6. The dilator of claim 1, further comprising a notch positioned between the tip and the coupling component.

7. The dilator of claim 6 wherein the notch is positioned between the first body region and the second body region.

8. The dilator of claim 6 wherein the notch is a first notch, the dilator further comprising a second notch positioned on a proximal or a distal side of the first notch.

9. A vascular access system, comprising:
a valve having a proximal end and a distal end;
a catheter defining a lumen and coupled to the valve; and
a dilator configured to be positioned within the lumen through the valve, wherein the dilator includes—
a first body region having a first flexibility profile; and
a second body region having a second flexibility profile less than the first flexibility profile,
wherein the first body region and the second body region have a same diameter.

10. The vascular access system of claim 9 wherein the first body region has a first stiffness, wherein the second body region has a second stiffness, and wherein the first stiffness is greater than the second stiffness.

11. The vascular access system of claim 9 wherein the first body region has a first durometer, wherein the second body region has a second durometer, and wherein the second durometer is less than the first durometer.

12. The vascular access system of claim 9 wherein the first body region comprises a first material, and wherein the second body region comprises a second material different than the first material.

13. The vascular access system of claim 9, wherein the dilator further includes a notch configured to extend at least partially around a longitudinal axis of the dilator, wherein the valve is configured to be releasably coupled to the dilator via the notch.

14. The vascular access system of claim 13 wherein the notch defines a recessed region of the dilator having a notch diameter less than the diameter of the first body region and the second body region.

15. The vascular access system of claim 13 wherein the valve includes a tether, and wherein the tether is configured to be positioned at least partially within the notch to releasably couple the dilator to the valve.

16. The vascular access system of claim 15 wherein the tether is biased in a first direction to releasably couple the dilator to the valve, the valve further comprising a button operably coupled to the tether and configured to move the tether in a second direction, opposite the first direction, to cause the tether to uncouple the dilator from the valve.

17. The vascular access system of claim 13 wherein the notch is a first notch, and wherein the dilator further includes a second notch positioned on a proximal side or a distal side of the notch.

18. The vascular access system of claim 17 wherein the catheter includes a distal terminus, wherein a first length of the dilator extends beyond the distal terminus when the first notch is coupled to the valve, and wherein a second length of the dilator extends beyond the distal terminus when the second notch is coupled to the valve.

19. The vascular access system of claim 18 wherein the second length is greater than the first length.

20. A method of treating clot material within a patient, the method comprising:
positioning a dilator within a vascular access system such that a valve of the vascular access system releasably engages a notch of the dilator to inhibit further movement of the dilator relative to the vascular access system;
actuating a button of the valve to release the notch from the valve to allow further movement of the dilator relative to the vascular access system; and
moving the dilator relative to the vascular access system and/or the vascular access system relative to the dilator.

21. The method of claim 20 wherein moving the dilator relative to the vascular access system includes advancing the dilator through the vascular access system to increase a length of the dilator that extends beyond a distal terminus of the vascular access system.

22. The method of claim 20 wherein moving the vascular access system relative to the dilator includes advancing the vascular access system over the dilator to decrease a length of the dilator that extends beyond a distal terminus of the vascular access system.

23. The method of claim 20 wherein the notch is a first notch, the method further comprising:
positioning the dilator within the vascular access system such that the valve releasably engages a second notch of the dilator; and
applying a force to the dilator and/or the vascular access system to release the second notch from the valve.

24. The method of claim 23 wherein applying the force to release the second notch from the valve includes applying the force to release the second notch from the valve without actuating the button.

25. A vascular access system, comprising:
a dilator extending along a longitudinal axis, wherein the dilator has a varying flexibility profile at least partially along the longitudinal axis, and wherein the dilator defines a notch extending at least partially circumferentially about the longitudinal axis;
a valve having a proximal end and a distal end; and
a catheter coupled to the valve and defining a lumen;
wherein the valve is configured to be releasably coupled to the dilator via the notch to inhibit movement of the dilator relative to the catheter when at least a portion of the dilator is inserted through the valve into the lumen of the catheter.

26. The vascular access system of claim 25 wherein the dilator includes a body region having a diameter, and wherein the notch includes a recessed region of the dilator having a notch diameter less than the diameter of the body region.

27. The vascular access system of claim 25 wherein the valve includes a tether, and wherein the tether is configured to be positioned at least partially within the notch to releasably couple the dilator to the valve.

28. The vascular access system of claim 27 wherein the tether is biased in a first direction to releasably couple the dilator to the valve, the valve further comprising a button operably coupled to the tether and configured to move the tether in a second direction, opposite the first direction, to move the tether at least partially out of the notch to uncouple the dilator from the valve.

29. The vascular access system of claim 25 wherein the notch is a first notch, wherein the portion of the dilator is a first portion, wherein the dilator further defines a second notch extending at least partially circumferentially about the longitudinal axis, and wherein the valve is configured to be releasably coupled to the dilator via the second notch to inhibit movement of the dilator relative to the catheter when at least a second portion of the dilator is inserted through the valve into the lumen of the catheter.

30. The vascular access system of claim 29 wherein the catheter includes a distal terminus, wherein a first length of the dilator extends beyond the distal terminus when the first notch is coupled to the valve, and wherein a second length of the dilator extends beyond the distal terminus when the second notch is coupled to the valve.

31. The vascular access system of claim 30 wherein the second length is greater than the first length.

32. The vascular access system of claim 25 wherein the dilator includes a proximal body region having a first flexibility profile and a distal body region having a second flexibility profile greater than the first flexibility profile, wherein the notch is positioned at least partially between the proximal body region and the distal body region.

33. The vascular access system of claim 25 wherein the dilator has a varying durometer at least partially along the longitudinal axis.
